(19) United States
(12) Patent Application Publication
(10) Pub. No.: US 2004/0018535 A1
(43) Pub. Date: Jan. 29, 2004

(54) Detection of RNA structural elements

(76) Inventors: Rangarajan Sampath, San Diego, CA (US); David J. Ecker, Encinitas, CA (US); Richard H. Griffey, Vista, CA (US); Gary B. Fogel, San Diego, CA (US); V. William Porto, Encinitas, CA (US)

Correspondence Address: COZEN O'CONNOR, P.C., 1900 MARKET STREET, PHILADELPHIA, PA, 19103-3508, US

(21) Appl. No.: 10/458,939

(22) Filed: Jun. 10, 2003

Related U.S. Application Data
(60) Provisional application No. 60/387,342, filed on Jun. 10, 2002.

(51) Int. Cl. C12Q 1/68; G06F 19/00; G01N 33/48; G01N 33/50

(52) U.S. Cl. 435/6; 702/20

(57) ABSTRACT

The present invention provides methods of identifying structures in nucleic acid sequences using evolutionary computation.

### CROSS-REFERENCE TO RELATED APPLICATIONS

This application claims priority to U.S. provisional application Serial No. 60/387,342 filed Jun. 10, 2002, which is incorporated herein by reference in its entirety.

### FIELD OF THE INVENTION

The present invention is directed, in part, to detection of RNA structural elements in RNA sequences using evolutionary computational methods.

### BACKGROUND OF THE INVENTION

RNA can be characterized by a base sequence and higher-order structural constraints. Short and long-range basepair interactions organize RNAs into secondary and tertiary structures required for biological function. Noncoding RNAs such as rRNA, tRNA, and other functional RNAs (e.g., RNase P, the signal recognition particle (SRP), and others) are highly structured. Secondary and tertiary structure is also likely to play an important role in mRNA regulation. For example, the iron responsive element (IRE) is a regulatory element located in the untranslated regions (UTRs) of mRNAs involved in iron metabolism and transport (Theil, Met. Ions Biol. Syst., 1998, 35, 403-434; Kim et al., J. Biol. Chem., 1996, 271, 24226-24230). Other mRNA secondary structures involved in processing and localization include stem-loops in the 3′-UTRs for histone and vimentin (Son, Saenghwahak Nyusu, 1993, 13, 64-70; Shepherd et al., Nucleic Acids Symp. Ser., 1997, 36, 142-145; Zehner et al., Nucleic Acids Res., 1997, 25, 3362-3370). In each of these examples, the structure is conserved even though the sequence has evolved over evolutionary history across a wide range of organisms. Given a hypothetical structure, a computational tool to mine sequence space for similar structures might lead to the discovery and understanding of novel functional and regulatory relationships.

RNA structures consist of bases that are either paired or unpaired. The majority of base pairings are of adjacent nucleotides forming antiparallel helices. The combination of helices and unpaired regions constitutes an RNA secondary structure. One way to approach the task of mining for conserved structural elements is to define the space of all structures that match a particular hypothesized motif and evaluate the presence or absence of these structures in a set of related RNA sequences. Computational tools have been developed to define and search for RNA secondary structure motifs including RNAMOT, Palingol, and PatScan (Gautheret et al., Comput. Appl. Biosci., 1990, 6, 325-331; Laferriere et al., Comput. Appl. Biosci., 1994, 10, 211-212; Billoud et al., Nucleic Acids Res., 1996, 24, 1395-1403; Pesole et al., Bioinformatics, 2000, 16, 439-450). Another search algorithm, RNAMotif, was introduced recently and provides the user with additional freedom to search for any definable simple or complex secondary and tertiary structure, including a variety of complex structural domains or non-canonical pairings that were not addressed by previous techniques (Macke et al., Nucleic Acids Res., 2001, 29, 4724-4735; Lesnik et al., Nucleic Acids Res., 2001, 29, 3583-3594). The structural patterns are defined by the user in a “descriptor” with a pattern language that gives detail regarding paring information, length, and sequence, providing a high degree of control over the structures that can be identified in a nucleotide sequence database. This tool can be used to generate a list of all possible structures that match a given descriptor within a set of sequences. Depending on the specificity of the descriptor and the number of nucleotides in the sequence database, this can result in a few hits or a very large number of hits (i.e., on the order of 10⁵ hits, or more, for a given bacterial genome). When the number of hits is large, exhaustive search for a set of maximally similar structures can be computationally infeasible. Attempts to make the search more feasible have been done using evolutionary algorithms, but ones used before the present invention have not been adequate.

Many independent efforts to stimulate evolution on a computer were offered as early as the 1950s and 1960s. Three broadly similar avenues of investigation in simulated evolution have survived as main disciplines within the field: evolution strategies, evolutionary programming, and genetic algorithms. These disciplines can be grouped in to the filed of evolutionary computation. The differences between the procedures are characterized by the typical data representations, the types of variations that are imposed to generate offspring, and the methods used to select parents (Fogel (ed.), Evolutionary Computation: The Fossil Record, 1998, IEEE Press, Piscataway, N.J.). The “no free lunch” theorem states in broad terms that all algorithms that do no resample points in a search space perform the same on average when applied across all possible functions (Wolpert et al., IEEE Trans. Evol. Compout., 1997, 1, 67-82). Therefore, no choice of variation operator, representation, or selection method can be uniformly superior over all problems. Previous attempts for RNA structure prediction using evolutionary computation have focused on genetic algorithms. Alternate representations and methods exist and have yet to be explored, not only for structure prediction (or “folding”) but also for calculation of RNA structure similarity. Fogel, “The application of evolutionary computation to selected problems in molecular biology” In:, Evolutionary Programming VI: Sixth International Conference, EP97, 1997, (Angeline, P. J., Reynolds, R. G., McDonnell, J. R. and Eberhart, R, eds.), Springer-Verlag, Berlin, Germany, pp. 23-33.

Typically, potential RNA structures have been examined by thermodynamic analysis accompanied by co-variation analysis based upon the alignment of nucleotide sequences. These types of analyses, however, place restraints on the information necessary to initiate an RNA structure query. Thus there is a long-felt need for improved methods to determine common structures found in RNA and other nucleic acid molecules using evolutionary computation to determine the structures, wherein the methods are not completely restricted by thermodynamic analysis and/or alignment analysis. The present invention fulfills this need as well as other needs for predicting and determining RNA structures.

### SUMMARY OF THE INVENTION

The present invention provides methods of detecting a conserved structure in an RNA sequence by: a) placing at least two structures from a plurality of structures generated for at least two RNA sequences from at least two organisms into a parent group, b) generating an offspring group from the parent group, wherein at least one structure is replaced in the parent group to generate the offspring group, c) determining fitness of the parent and offspring groups, d) comparing the fitness of the parent and offspring groups, and e) selecting at least one group from the parent and offspring groups with the highest fitness, wherein the conserved structure in the RNA is present within the at least one group. Steps b)-e) can be repeated iteratively. The iterations can be stopped by a user-defined criteria, such as, for example, number of generations, CPU time, clock time, or use of a statistical method to determine the appropriate number of generations. A representative statistical method that can be used can be determining when the expected change in fitness per generation is close to zero and past which further computation will not result in a large change in fitness but will take an unreasonable amount of time.

In some embodiments, the replaced structure can be replaced with a structure from a different organism or from the same organism. The parent group can comprises B structures, wherein B is greater than one and less then or equal to the number of structures that were generated. The parent group can comprises B structures, wherein B is equal to the number of organisms. The parent group can also comprises at least one structure from each organism. At least two structures can be replaced in the parent group to generate the offspring group. The parent and offspring groups can also be treated as one evolving population.

In some embodiments, the plurality of RNA structures can be generated using RNAMotif. Comparing the fitness of the groups can be carried out by an elitist selection or by a tournament selection.

### BRIEF DESCRIPTION OF THE DRAWINGS

FIG. 1 shows an example RNAMotif output (top) containing structures found in three organisms. The sequence ID is the GenBank accession number (gi). During initialization of the evolutionary algorithm, each parent bin P is constructed by selecting B structures at random from the RNAMotif output file. Depicted here is the case where P=2 and B=5.

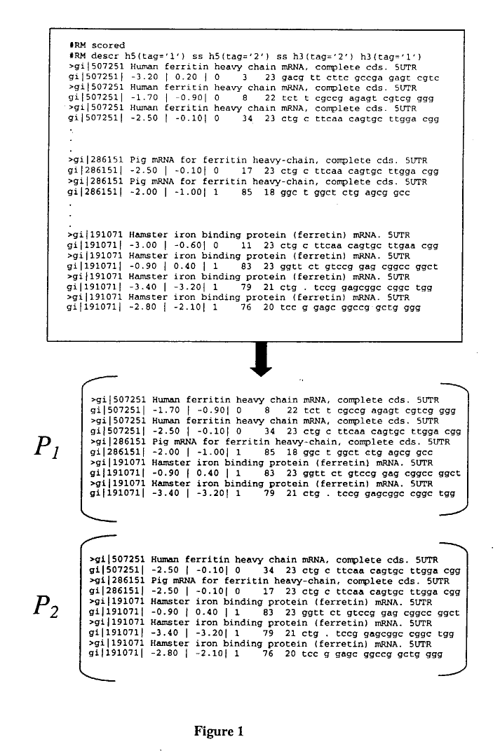

FIG. 2a shows a structure replacement within a specified sequence ID variation operator. A parent bin P is chosen at random to generate an offspring bin O. Within P, a number of structures (in this case 1) are chosen at random for replacement (bold italics). A new structure from the same sequence ID is pulled from the RNAMotif output file ensuring that bin O has no identical structures.

FIG. 2b shows a structure replacement within a different sequence ID variation operator. A parent bin P is chosen at random to generate an offspring bin O. Within P, a number of structures (in this case 1) are chosen at random for replacement (bold italics). A new structure from a different sequence ID is pulled from the RNAMotif output file ensuring that bin O has no identical structures. In this case the hamster structure is replace with one from pig.

FIG. 2c shows a random single-point bin recombination operator. The information in two parent bins (one from the evolving population and the other generated at random) is exchanged to form two offspring bins (O1 and O2) about a single point of recombination. Either O1 or O2 is selected at random to become a new member of the population. Random multi-point bin recombination makes use of the same basic procedure except with multiple points of recombination.

FIG. 3 shows a nucleotide association matrix for scoring pairwise threaded nucleotide similarity. Any nucleotide symbol paired against a gap (−) will receive an initial gap opening penalty of −12. In sequence alignments of RNAMotif structures, it is possible to have the condition where a gap can be paired with a gap. In this case, the position is given a score of 0.

FIG. 4a shows an overview of pairwise sequence similarity scoring. Two structures in a bin are chose for pairwise nucleotide alignment. Components (h5, ss, h3) without any sequence in the structure (i.e. a missing bulge; represented as “.”) are treated as gaps during sequence alignment. Using the scoring matrix in FIG. 3, scores (ss) are generated for each component block (numbered 1-7) of the structure. User-defined weights (CW) are also associated with each component and the sums for these values are determined. The sequence scores are multiplied by the component scores to generate a weighted score (SS′). A final SEQ score is generated by dividing the sum of the weighted score by the sum of the component weights and dividing by the length of the longest structure.

FIG. 4b shows an overview of pairwise structure similarity scoring. Two structures in a bin are chosen for pairwise nucleotide alignment. Components (h5, ss, h3) without any sequence in the structure (i.e. a missing bulge; represented as “.”) are maintained during structure alignment. Scores (ST) are generated for each component block (numbered 1-7) of the structure. User-defined weights (CW) are also associated with each component and the sums for these values are determined. The structure scores are multiplied by the component scores to generate a weighted score (ST′). A final SCLS score is generated by dividing the sum of the weighted score by the sum of the component weights. For structure comparison, a “.” symbol is given a value of 0.

FIG. 4c shows an overview of pairwise structure thermodynamic stability similarity score. Two structures in a bin are chose for pairwise efn scoring. For each structure in the case above, two efn scores (EFNₐ and EFN_{b}) are provided in the RNAMotif output file for different portions of each structure. The similarity between these values is compared pairwise, where efn similarity is maximized. The similarities are summed and divided by the number of efn components to generate a final EFN score for the pair.

FIG. 5 shows a schematic overview showing the evolutionary algorithm for structural element similarity.

FIG. 6 shows structures of the human ferritin iron-responsive element (a, b) and structure of human SRP domain IV (c) found in the literature.

FIG. 7 shows an RNAMotif descriptor used for ferritin experiment 1, based loosely on the known structures for the ferritin IRE.

FIG. 8 shows top five results from experiment 1 (Example 1) with ferritin mRNA using the evolutionary computation from generation 13 ranked in order of decreasing fitness. Bin #1 at the top is identical to the known correct ferritin IRE structure. Bins are represented as a series of structures, one chosen from each of 7 different organisms. Information includes the sequence ID (gi|#), position of the hit within the ferritin mRNA for that gi record, the length of the structure, and the strand (0=sense, 1=antisense).

FIG. 9 shows an RNAMotif descriptor used for ferritin experiments 2 and 3, with less restriction on length in the upper stem.

FIG. 10 shows top five results from experiment 2 (Example 1) with ferritin mRNA using evolutionary computation from generation 33 ranked in order of decreasing fitness. Bin #1 at the top is identical to the known correct ferritin IRE structure. Bins are represented as a series of structures, one chosen from each of 7 different organisms. Information includes the sequence ID (gi|#), position of the hit within the ferritin mRNA for that gi record, the length of the structure, and the strand (0=sense, 1=antisense).

FIG. 11 shows top five results from experiment 3 (Example 1) with ferritin mRNA using evolutionary computation from generation 21 ranked in order of decreasing fitness. Bin #1 at the top is identical to the known correct ferritin IRE structure. Bins are represented as a series of structures, one chosen from each of 12 different organisms. Information includes the sequence ID (gi|#), position of the hit within the ferritin mRNA for that gi record, the length of the structure, and the strand (0=sense, 1=antisense).

FIG. 12 shows RNAMotif descriptor used for ferritin experiment 4 (Example 1). This descriptor has unpaired nucleotides on the 3′ side of the stem in opposition to the original bulge. This descriptor provides the possibility for internal loops in the final products.

FIG. 13 shows top five results from experiment 4 (Example 1) with ferritin mRNA using evolutionary computation from generation 115 ranked in order of decreasing fitness. Bin #1 at the top is identical to the known correct ferritin IRE structure. Bins are represented as a series of structures, one chosen from each of 7 different organisms. Information includes the sequence ID (gi|#), position of the hit within the ferritin mRNA for that gi record, the length of the structure, and the strand (0=sense, 1=antisense).

FIG. 14 shows an RNAMotif descriptor used for SRP experiment 1 (Example 2). This descriptor is a very close description of the known SRP structure.

FIG. 15 shows top five results from experiment 1 (Example 2) with SRP using evolutionary computation from generation 3 ranked in order of decreasing fitness. Bin #1 at the top is identical to the known correct ferritin IRE structure. Bins are represented as a series of structures, one chosen from each of 5 different organisms. Information includes the sequence ID (gi|#), position of the hit within the SRP for that gi record, the length of the structure, and the strand (0=sense, 1=antisense).

FIG. 16 shows an RNAMotif descriptor used for SRP experiment 2 (Example 2). Increased length variability was introduced to the lower stem.

FIG. 17 shows top five results from experiment 2 (Example 2) with SRP using evolutionary computation from generation 7 ranked in order of decreasing fitness. Bin #1 at the top is identical to the known correct ferritin IRE structure. Bins are represented as a series of structures, one chosen from each of 7 different organisms. Information includes the sequence ID (gi|#), position of the hit within the SRP for that gi record, the length of the structure, and the strand (0=sense, 1=antisense).

FIG. 18 shows an RNAMotif descriptor used for SRP experiment 3 (Example 2). Increased length variability was introduced to both stems relative to experiment 1.

FIG. 19 shows top five results from experiment 3 (Example 3) with SRP using evolutionary computation from generation 27 ranked in order of decreasing fitness. Bin #1 at the top is identical to the known correct ferritin IRE structure. Bins are represented as a series of structures, one chosen from each of 5 different organisms. Information includes the sequence ID (gi|#), position of the hit within the SRP for that gi record, the length of the structure, and the strand (0=sense, 1=antisense).

FIG. 20 shows an RNAMotif descriptor used for SRP experiment 4 (Example 2). Increased length variability was introduced to both stems and all single stranded regions relative to experiment 1 of Example 2.

FIG. 21 shows top five results from experiment 3 (Example 3) with SRP using evolutionary computation from generation 27 ranked in order of decreasing fitness. Bin #1 at the top is identical to the known correct ferritin IRE structure. Bins are represented as a series of structures, one chosen from each of 5 different organisms. Information includes the sequence ID (gi|#), position of the hit within the SRP for that gi record, the length of the structure, and the strand (0=sense, 1=antisense).

### DESCRIPTION OF EMBODIMENTS

The present invention provides methods of determining and/or identifying common structural elements of a nucleic acid molecule. Nucleic acid molecules include DNA and RNA. The structural elements may be in the form of nucleic acid molecules isolated from a cell or virus, or may be in the form of synthetic nucleic acid molecules, such as oligomers, and, in particular, oligonucleotides. Cells include, for example, eukaryotic and prokaryotic cells and include, but are not limited to, bacterial cells, fungal cells, protozoan cells, and mammalian cells.

In some embodiments a plurality of RNA sequences is analyzed to generate a plurality of structures for each RNA sequence. The structure search can be carried out using any method that generates a plurality of structures for a particular sequence. For example, RNAMotif can be used to produce a list of structures (or “hits”) that conform to a particular structure descriptor. RNAMotif is described in, for example, Nucleic Acids Research, Nov. 15, 2001, 29(22), 4724-35 and can be accessed at, for example, ftp.scripps.edu/pub/macke/rnamotif-2.4.0.tar.gz. Additional computer programs can also be used, such as RNAstructure and the like. The RNAMotif output file can contain the following information: structure pairing information, a sequence identifier, the position of a hit relative to the start of the sequence, the number of nucleotides in the structure, the strand (sense or antisense), and nucleotide sequence associated with the RNA structure. The information contained in the RNAMotif output serves as input to the evolutionary algorithm.

To generate an initial population of contending solutions, a collection or “bin” of structures is chosen at random without replacement from the space of all structures represented in the RNAMotif output file. Each bin represents one contending solution in the initial population, and is referred to as a “parent bin” for the initial generation of evolution. In some embodiments, the initialization process is repeated until P parent bins are created. As used herein, the term “P” refers to a number of parent bins that is a user-defined parameter. In some embodiments at least 2, at least 5, at least 10, at least 20, at least 30, at least 40, at least 50, at least 75, at least 100, at least 1000, at least 2000, at least 5000, or at least 10,000 parent bins are created. The number of structures, or hits, contained in each bin is referred to as the “bin size” “B”, where B is also a user-defined parameter. In some embodiments, both B and P are fixed throughout one run of evolution.

During initialization, each of P bins is constructed by selecting B structures at random from the RNAMotif output file, where 1<B≦Bₘₐₓ (Bₘₐₓ=the total number of structures in the RNAMotif output file). In some embodiments, when B is larger than the number of organisms represented in the RNAMotif output file, multiple structures for a given sequence ID may occur. In some embodiments, a person of ordinary skill in the art can define a parameter to force only one structure to be drawn at random from each sequence ID.

Variation can be carried out by a number of procedures, one of which is described as follows. For the initial generation, O “offspring bins” are generated from a P bin or group, where O is a user-defined parameter (e.g., an integer defining the desired number of offspring bins). In some embodiments, once O offspring bins have been generated, the parent and offspring bins are treated as one evolving population. During this generation process, variation operators are applied so that each offspring will have some difference relative to the parent. In some embodiments, a first random variable is drawn from a user-specified probability distribution (e.g. Poisson or Gaussian) to determine which of the variation operators described herein are chosen. A second random variable can also be drawn from a user-defined probability distribution to determine the number of times a particular variation operator is applied to the parent bin when generating an offspring. In some embodiments, the possible variation operators include, but are not limited to, 1) structure replacement within a specified sequence ID, 2) structure replacement from a different sequence ID, 3) random single point-bin recombination, and 4) random multi-point bin recombination.

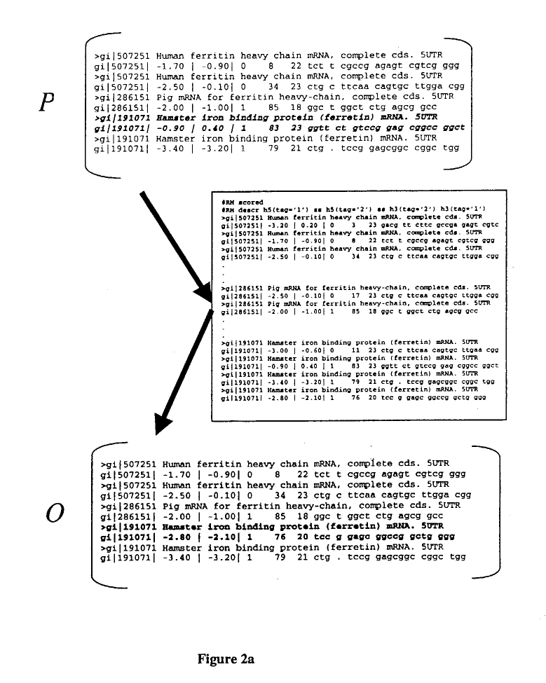

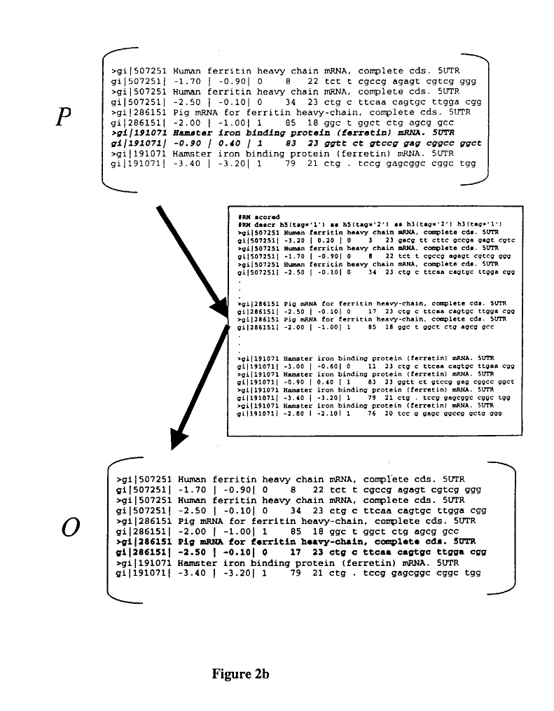

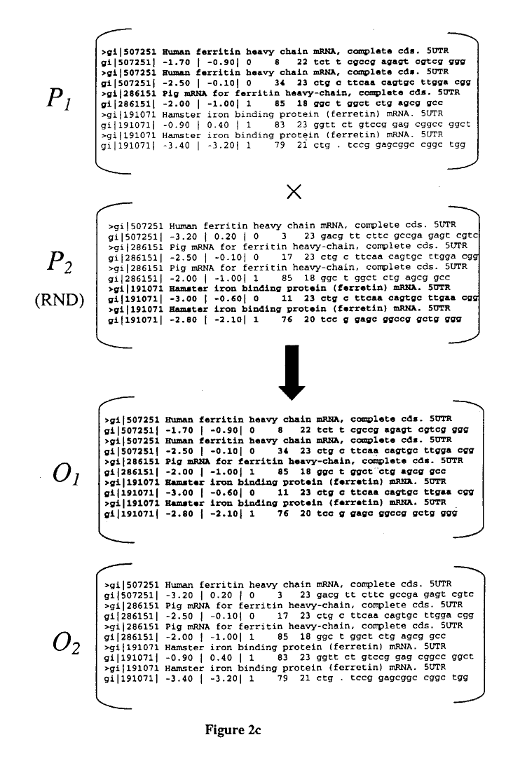

FIGS. 2a-c demonstrate each of the possible variation operators. For the structure replacement within a specific sequence ID operator (type 1), structures in a bin are replaced at random with new structures from the same organism in the RNAMotif file (FIG. 2a). For example, in this embodiment, the bin size is set to five, but can be any number greater than one. With this operator, a new set of random variables is required. A first random variable is chose to determine which of the five structures is replaced with a minimum number of replacements (i.e. 1) or maximum of B−1. A second random variable can also be selected to determine between two choices of a range (local or global) for the difference in structural similarity between the old and new structures. RNAMotif output files contain structures that are listed in order of position relative to the 5′ end of the target sequence. Therefore, within the file, neighboring RNAMotif structure hits have a higher probability of structural similarity than do hits found at large distances over the file. The local version of the structure replacement within a specified sequence ID operator chooses a replacement structure from the RNAMotif file that neighbors the original structure in the file. This mutation will have a better-than-average chance to return a structure that is quite similar to the original structure due to the organization of the RNAMotif output file. The global version of the structure replacement within a specified sequence ID operator chooses a replacement structure at random from the RNAMotif file without replacement. The global version of this variation operator allows for the possibility of large jumps in the structure space represented by the RNAMotif file whereas the local variation operator provides jumps that have a higher probability of returning a similar structure from the RNAMotif file.

The structure replacementfrom a difference sequence ID variation operator (type 2) is used to randomly replace a structure in a bin with a new structure from a different organism in the RNAMotif file (FIG. 2b). For example, if hits from 10 different organisms in the RNAMotif file and a bin size of 5 (B=5). A random number is drawn for the number of structures to be replaced in the bin, with a minimum and maximum number of replacements of 1 and B−1 respectively. If one structure is chosen for replacement, a new structure is chosen at random from the set of structure hits in one of the other sequence IDs in the RNAMotif file. In the event or embodiments that the user has indicated that only one structure from each organism is to be used in the bin, the random sampling is only from organisms not yet represented in the bin.

The random single-point bin recombination operator (type 3, shown in FIG. 2c) makes use of the information in two parent bins to generate two new offspring bins via single-point recombination. When using the random single-point bin recombination operator, one parent bin (P₁) is selected at random from the population whereas the other parent bin (P₂) is a newly constructed random draw of structures from the RNAMotif file. For example, assuming a bin size of 5 (B=5), within P₁, a random variable is used to select a structure, for example, structure 3. Structure 3 would then serve as a position of single-point recombinant between P₁ and P₂, to generate two new offspring binds, O₁ and O₂. O₁ would contain the first 3 structures from P₁ and the last 2 structures from P₂. One of the two possible offspring (O₁ and O2) is selected at random to become a new member of the population. During the evolutionary process, this operator therefore combines a parent bin containing implicit evolutionary history (P₁) with a new parent bin (P₂) constructed completely at random in order to allow for very large jumps across the search space. The random multi-point recombination operator (type 4) makes use of the same basic procedure except with multiple points of recombination.

A process of self-adaptation (Fogel, Evolutionary Computation: Toward a New Philosophy of Machine Intelligence, 2nd Edition, 2000, IEEE Press, Piscataway, N.J.) can be used to tune the probabilities associated with each variation operator concurrently with the process of evolution. Every solution in the population carries its own set of variation probabilities and passes the information to the subsequent generation. The number of variation operators applied to each individual is determined by the formula

Y′ₖ₊₁=Yₖ^{\hat{ }}+N(0,1)

where Yₖ is the mean number of variation operators to apply at step k and N(0, 1) is a normal Gaussian distribution with mean μ=0, and standard deviation σ=1. Given U structures in a bin,

Yₖ₊₁=max(min(Y′ₖ₊₁, U/2),1)

The actual number of variation operators (Qₖ) to apply at step k can be generated using a Poisson distribution with mean Yₖ

Qₖ=Poisson(Yₖ)

Given a specific value for Qₖ, variation operators must now be identified. This choice can be made as a probability over the six possible variation operators.

Let m=the number of possible variation operators

ρ=1/m

γ=0.1×ρ

αε[0,1]

βε[0,1]

α_{k,1}=probability of choosing operator i at time step k where α_{k,i}ε[0,1]∀, and

Let

$$\begin{matrix} & {\sum }_{i = 1}^{n} {a}_{k , i} = 1 \\ Let & {d}_{k , i} = ( {a}_{k , i} - ρ ) \\ & {\hat{d}}_{k , i} = \vert {d}_{k , i} \vert \\ & {x}_{k , i} = m \times {a}_{k , i} + α [ N ( 0 , 1 ) - β \times {\hat{d}}_{k , i} \times sign ( {d}_{k , i} ) ]\end{matrix}$$

where

sign(x)=1 if x≧0

sign(x)=−1 if x<0.

The factor m applied to α_{k,i} scales x_{k,i} into the range [0,1]+epsilon. The term β×\hat{d}_{k,i}×sign(dₖ) is proportional to the difference between the current (at time step k) probability of choosing operator i and a uniform probability of choosing from the m operators. Thus this term acts to drive α_{k,i} back toward a uniform distribution. Finally,

\hat{x}_{k,i}=max(x_{k,i},γᵢ)

where γ represents a minimum probability threshold value to ensure the probability of choosing operator i is always non-zero and rescaling.

$${a}_{k + 1 , i} = \frac{{\hat{x}}_{k , i}}{{\sum }_{i = 1}^{n} {\hat{x}}_{k , i}}$$

In addition to these methods of generating bins, a person of ordinary skill in the art can choose to either allow or avoid the placement of structures that overlap in sequence space in the same bin. This filter can be used to avoid trivially redundant, yet similar structures, from collecting in bins.

Fitness can also be carried out by many procedures. An exemplary procedure is described below. An application of an evolutionary algorithm can be used to search for the most similar structural elements in an RNAMotif output file. Sets of structures (bins) are pulled from the RNAMotif file. Evolutionary computation is used to search the large space of possible bins to find the bin of maximum similarity. For this purpose, a measure or score in the form of a fitness function is used such that a bin or bins containing most similar structures are given a higher fitness or score and therefore a higher probability of survival to the subsequent round of evolution. The fitness function is an aggregate of components that measure RNA structure similarity. These measures are applied pairwise by each structural component and then summed into a final score representing the fitness for each bin. The scoring components include but are not limited to 1) nucleotide sequence similarity within a structural component, 2) structure component length similarity, and 3) structure thermodynamic stability similarity.

The nucleotide sequence similarity within a structural component fitness function was designed under the hypothesis that, in RNA, structure is conserved over sequence. For example, given a set of diverse organism, several domains of 16S rRNA have a common structure. However, within structural sub-domain, different nucleotides and base pairings might have naturally evolved over time to preserve certain overall secondary and higher-order interactions. Gutell et al., J. Mol. Biol., 2000, 304, 335-354;Gutell et al. J. Mol. Biol., 2000, 300, 791-803; Cannone et al., BMC Bioinformatics, 2002, 3, 2. Comparison of nucleotide sequence information found in an RNA structural element is most informative when an alignment is first based on structural information.

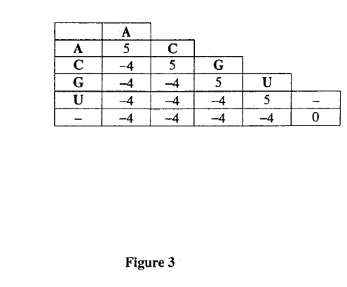

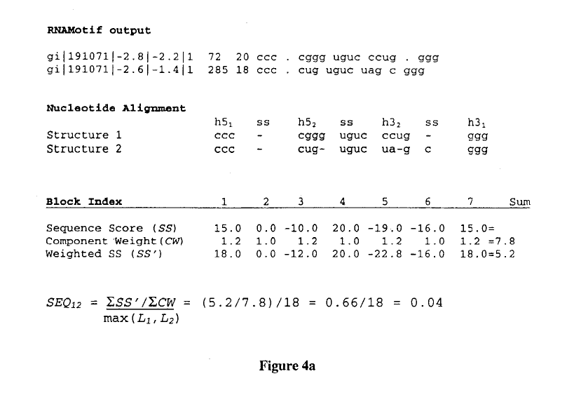

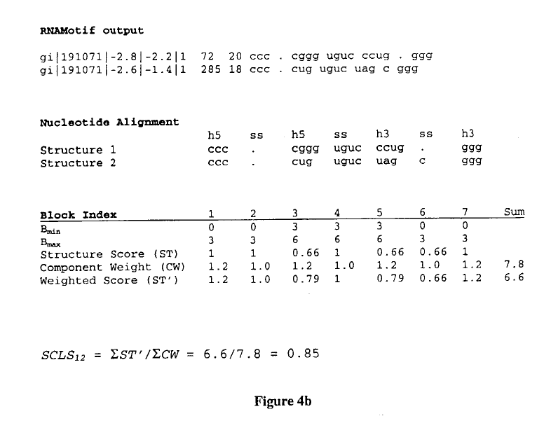

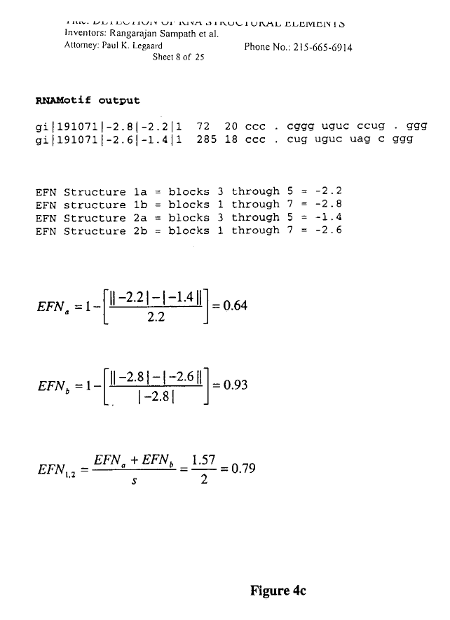

Using the algorithm ALIGN (Myers et al., Comput. Appl. Biosci., 1988, 4, 11-17), the nucleotide strings representing different structures in a single bin are compared in a pairwise fashion to generate the best pairwise alignments over the set of symbols (A, G, C, U, −) using the match, mismatch, and gap penalty values shown in FIG. 3. Pairwise alignment scores at the nucleotide level are calculated with reference to the structure components in the associated RNAMotif descriptor. For example, given an RNAMotif output file for a descriptor of a simple stem-loop (hs ssh3) (Macke et al., Id.), nucleotides on the 5′ side of the stem (h5) are scored pairwise as a structure component block, followed by alignment of the nucleotides in the loop (ss), and a third alignment of the nucleotides on the 3′ side of the stem (h3). All component blocs (b) are scored separately for pairwise nucleotide similarity and associated with a sequence score (SS_{b}) (FIG. 4a).

Component-based calculation of similarity offers distinct advantages in that a person of ordinary skill in the art may, in some embodiments, specify an additional bonus for similarity in a particular structural component (e.g., nucleotide similarity in the loop region may carry more importance than nucleotide similarity in the stem). In the example presented in FIG. 4a and in examples described herein, stems were given a weight of 1.2 and single strand regions a weight of 1.0. The weights associated with all components in the pairwise comparison are summed for an overall score (CWₜₒₜ) using the equation

$${CW}_{tot} = {\sum }_{b = 1}^{n} {CW}_{b}$$

where b is the index for each component block. A weighted sequence score is then generated for each block

SS′_{b}=CW_{b}×SS_{b}

and the weighted score is summed over all blocks

$${SS}_{tot}^{i} = {\sum }_{b = 1}^{n} {SS}_{b}^{′}$$

A final pairwise sequence score (SEQ) is generated using the equation

$${SEQ}_{i , j} = \frac{( {SS}_{tot}^{i} / {CW}_{tot} )}{max ( {L}_{i} , {L}_{j} )}$$

where i and j are structure indices and L is the length of the sequences being compared. An example of this calculation is shown in FIG. 4a.

The above calculation represents the sequence comparison of two structures in a bin. The overall fitness score for the sequence similarity of all structure pairs in a bin can be calculated by summing the SEQ_{i,j} scores and normalizing this value over the number of pairwise combinations (p) in a bin

$${SEQ}_{tot} = \frac{{\sum }_{i = 1}^{n} {\sum }_{j = 1}^{n} {SEQ}_{i , j}}{p}$$

The range of minimum and maximum possible alignment scores in the RNAMotif output file is then calculated. This can be carried out by, for example, determining the longest sequence for each structure block in the output file, and calculating scores for the theoretical conditions where each of the longest structure block was either paired with an identical copy of this sequence (the maximum sequence similarity score over the entire RNAMotif file), or with an equally long artificial “sequence” composed only of gaps (maximal dissimilarity). These maximum and minimum scores were used for normalization to the range [0,1] for all other sequence comparisons. Each bin score was placed in this range using the equation

$${SEQ}_{tot}^{′} = [ {SEQ}_{tot} \times ( \frac{b - a}{d - c} ) ] + ( \frac{a \times d - b \times c}{d - c} )$$

where a=0, b=1, c is the maximal dissimilarity score in the RNAMotif output file, d is the maximal similarity score in the RNAMotif output file and SEQₜₒₜ is the total sequence score for all pairwise comparisons in a bin.

The second term in the fitness function, structure component length similarity (SCLS), is used to measure the similarity in terms of the lengths of all components in a structure. In the case, where a range of lengths is provided for any structure component in an RNAMotif descriptor, components of differing lengths can be generated. For each structure being compared, the length of each component is determined. The lengths of these individual structural components are compared on a pairwise basis for all structures in a bin. A structure score for each component block (ST) is calculated using the equation

$${ST}_{b} = 1 - [ \frac{max ( {C}_{1 b} , {C}_{2 b} ) - min ( {C}_{1 b} , {C}_{2 b} )}{max {B}_{b} - min {B}_{b}} ]$$

where C₁ and C₂ are the structures being compared, max(C_{1b}, C_{2b}) is the maximum length sequence within component block b, min(C_{1b}, C_{2b}) is the minimum length sequence within block b, max B_{b} is the maximum length structure for block b found in the RNAMotif output file, and min B_{b} is the minimum length structure for block b found in the RNAMotif output file. In the condition where two structures contain missing components (represented as “.” in RNAMotif), each component is equated with a score of 0.

User-defined weights are associated with the importance of similar length for each structure component. For the experiments described herein, these weights were all set to 1.0, except for length similarity in stems, which was set to 1.2. The component weight scores are summed over all component blocks b using, for example, the formula

$${CW}_{tot} = {\sum }_{b = 1}^{n} {CW}_{b}$$

A weighted sequence score is then generated for each block

ST_{b}=CW_{b}×ST_{b}

and the weighted scored summed over all blocks

$${ST}_{tot} = {\sum }_{b = 1}^{n} {ST}_{b}^{′}$$

The pairwise structure component length similarity scores are summed to form an overall structure component length similarity score for each pair, with the sum then normalized over the number of blocks in the structure. An equation for this calculation is given by, for example,

$${SCLS}_{i , j} = \frac{{ST}_{tot}}{{CW}_{tot}}$$

To determine an overall score for a bin in terms of structure similarity, the SCLS scores are summed over all pairwise comparisons and normalized by the number of pairwise comparisons in the bin.

$${SCLS}_{tot}^{′} = \frac{{\sum }_{i = 1}^{n} {\sum }_{j = 1}^{m} {SCLS}_{i , j}}{p}$$

Depending on the descriptor format, portions of the structures (and/or the entire structures) in the RNAMotif output file may contain scores for thermodynamic stability calculated using the function efn (Matthews et al., J. Mol. Biol., 1999, 288, 911-940). When evaluating structure similarity using our algorithm, these efn values can also be used for structure comparison. To derive a structure thermodynamic stability similarity score (EFN) for a pair of structures, the difference in efn values between two portions of an overall structure can be calculated and divided by the maximum of the two values (FIG. 4c).

$${EFN}_{s} = 1 - \frac{\vert ( {efn}_{1 s} - {efn}_{2 s} ) \vert}{max ( \vert {efn}_{1 s} \vert , \vert {efn}_{2 s} \vert )}$$

where s is the portion of the structure receiving an efn score, efn₁ is the score for structure 1, efn₂ is the score for structure 2. Our fitness function minimizes the difference in efn value. Comparisons of structure components form an efn score (EFNₜₒₜ) for the pair, and are normalized over the number of portions in the structure receiving an efn score

$${EFN}_{i , j} = \frac{{\sum }_{s = 1}^{n} {EFN}_{s}}{s}$$

where s is the number of portions receiving an efn score, i and j are structure indices. To determine a total EFN score for a bin, the EFN scores for all pairwise combinations can be summed and dived by the number of pairwise combinations

$${EFN}_{tot}^{′} = \frac{{\sum }_{i = 1}^{n} {\sum }_{j = 1}^{m} {EFN}_{i , j}}{p}$$

These three fitness terms are combined to form a value representative of the overall worth of a given bin. The importance of each of these fitness terms is associated with a weight that can be user-defined. The total fitness (F_{bin}) of any given bin is therefore defined as the sum of its weighted component scores

$${F}_{bin} = \frac{{w}_{1} ( {SEQ}_{tot}^{′} ) + {w}_{2} ( {SCLS}_{tot}^{′} ) + {w}_{3} ( {EFN}_{tot}^{′} )}{{w}_{1} + {w}_{2} + {w}_{3}}$$

where wᵢ are the weights associated with the terms sequence alignment (SEQ′ₜₒₜ), structure component length similarity (SCLS′ₜₒₜ) and stem efn similarity (EFN′ₜₒₜ). In some embodiments, and in the examples described herein, SEQ was weighted slightly more than SCLS and efn was ignored (w₁=0.8, w₂=0.2, w₃=0.0).

Based on the fitness cores, a mechanism of selection is required to determine which bins will be removed from the current population (and, by consequence, which remaining bins will serve as parent bins for the next generations). Two methods of selection can be used for this purpose, although any other method of selection is also suitable. A person of ordinary skill in the art can decide at the beginning of the method which selection procedure is appropriate.

Under an elitist selection approach (Fogel, (2000) Id.), all bins in the population are ranked with respect to their fitness score. The top X bins from this rank ordered list are saved to become parents for the next generation. As used herein, the term “X bins” refers to a number of bins defined by a person of ordinary skill in the art that are saved. In some embodiments at least 2, least 5, at least 10, at least 50, at least 100, or at least 1000 bins are saved to become parents for the next iteration. The bins that are not saved are discarded.

Under a tournament selection approach (Fogel, (2000) Id.), a bin from the current population is chosen at random and is “competed” with a set of R randomly chosen bins in the same population, where R is a user-defined parameter. Each time the first bin's fitness score is higher than (or ties) the opponent's score, the first bin receives a “win.” The number of wins is recorded for all competitions and this process can be iterated over all members of the population. All bins are then ranked with respect to the number of wins received during the competition. Selection is then used to remove the lower Z bins on this ranked list, where Z is a user defined parameter and in some embodiments Z is at least 1, at least 5, at least 10, at least 50, at least 100, or at least 500. In the case of a tie in this ranking of wins and losses, those specific bins can be re-ranked by their fitness score prior to selection. After selection, the remaining Q bins are saved to serve as parents for the next generation. In some embodiments, Q refers to all the remaining bins, and in other embodiments Q refers to the 2 top ranked, 5 top ranked, 10 top ranked, 50 top ranked, 100 top ranked, or 500 top ranked bins.

The effect of these two selective mechanisms is different depending on the complexity of the space that is being searched. Given a monotonically decreasing continuous function, elitist selection will quickly drive the population towards a global optimum by saving the best solutions at each generation. However, many biological problems (such as the ones described below) can be discontinuous, multimodal, and noisy. In these cases, it can be more efficient to ensure the low probability that some solutions of lesser worth survive to subsequent generations through a tournament selection approach. These solutions may later be beneficial in escaping future local optima to avoid premature convergence.

With the application of selective mechanism, the first generation of evolution is completed. The Q saved bins from the first round of selection are used as “parents” to generate offspring bins with variation in the manner described above. All parent and offspring solutions are pooled into a single population, scored, and selected in the manner described above. In some embodiments, this process is iterated until user-specific termination criteria are satisfied. These criteria may include, but not limited to, a user defined number of generations, CPU or clock time, or use of statistical methods (to determine the appropriate number of generations when the expected change in fitness per generation is close to zero and past which further computation will not result in a large change in fitness but will take an unreasonable amount of time). An unreasonable amount of time may be from about 10 minutes or longer, about 30 minutes or longer, about one hour or longer, about 6 hours or longer, or more than one day or longer. A large change in fitness is about 10%, about 1%, about 0.1%, or about 0.01% change in fitness. “About” means±5% of the value it modifies. The number of total function evaluations (Eₜₒₜ) during the evolutionary process (Fogel, (2000) Ibid.) can be calculated as Eₜₒₜ=(P×O×G)+P, where P is the number of parent bins, O is the number of offspring bins, and G is the number of generations.

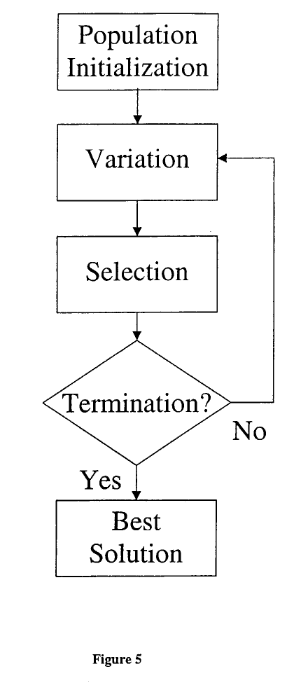

In the examples presented below, a rule that no two bins in the population at any single generation may share identical structure sets was applied. However, this is not required. Therefore, the final generation of evolution contained only unique bins rank-ordered by fitness. A schematic overview of the entire evolutionary algorithm is provided in FIG. 5.

In order that the invention disclosed herein may be more efficiently understood, examples are provided below. It should be understood that these examples are for illustrative purposes only and are not to be construed as limiting the invention in any manner. All of the evolution work shown here was performed in parallel on 3-5 dual processor Intel Pentium 3, 450 Mhyz, 256 MB RAM computers, running Linux O/S using server/client architecture, although other computer systems can be used. A “master” server program serves as the user interface, reading parameters user inputs, and RNAMotif data files. This program then spawns one or more “client” programs that perform the actual evolution process. Each client starts the evolution with a unique random number seed, periodically transmitting its best solution set back to the master program. Although the clients typically act as parallel evolutionary “islands,” data can be communicated between clients, hence they can augment their current solution set with information sent from other client processes. This sharing of evolved information between clients facilitates escape from local optima points and improves the rate of convergence significantly.

For all the examples described herein, tournament selection, Poisson probability distributions for the number of mutations, and self-adaptation using Gaussian distribution were used with varying population sizes for 1000 generations of evolution on four Linux machines operating in parallel. The time to convergence on the known solutions for these RNA structures was measured and the remainder of evolution was monitored to ensure that “better” solutions were not generated. The data presented below can also be found in Fogel et al. (Nucleic Acids Research, 2002, Vol. 30, No. 23, 5310-5317, which is incorporated herein by reference in its entirety.)

In some embodiments, the following user-controlled parameters and options were used. Evolution parameters: −12344/random seed, must be negative (−1234); 100/number of parents in the population (3); 50/number of offspring per parent (2); and 1000/number of generations (100). Variation parameters: 8.000000/mean number of mutations (1.000000); 1/minimum number of mutations (1.00000); 1/Type of mutation strategy (Poisson −1, Standard −0); and YES/do you want to use self-adaptation (YES). Selection parameters: NO/Do you want to use tournament selection (default ELITIST) (NO); 100/Number of tournament contests (10); and NO/Permit re-drawing in tournament contestants (NO). Scoring weights for fitness function in evolution: 0.600000/nucleotide Alignment Score Weight (0.500000); 0.400000/homology Score Weight (0.500000); and 0.000000/component EFN during Selection (0.000000). Output options: 5/checkpoint interval to save results (10); and 10/Number of solutions to report at the end of evolution (5). EFN on entire structure—not currently being used. Using RNAMotif EFN values: NO/EFN after Evolution (NO); and 20.000000/total EFN Weight after Evolution (0.000000). Termination options: YES/Would you like to terminate with generations (YES); NO/Would you like to terminate with Wall Clock time (NO); 0/value at which you would like to terminate with Wall Clock time (0); yes/Would you like to terminate with curve fitting (No); and 500/Generation number to allow program to run without a change in fitness (50).

### EXAMPLES

### Example 1

Motif Searching

Iron-Responsive Element (IRE).

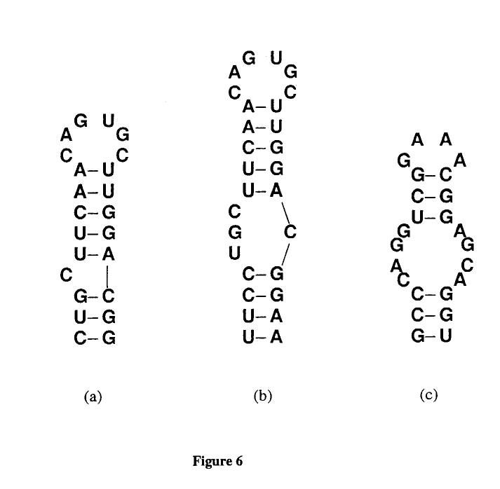

IREs have been described in the 5′- and 3′-UTRs of several mRNAs (Thiel (1998) Ibid., Ke et al., Biochemistry, 2000, 39, 6235-6242; McKie et al., Mol. Cell, 2000, 5, 299-309; Thomson et al., Int. J. Biochem. Cell Biol., 1999, 31, 1139-1152; and Schlegl et al., RNA, 1997, 3, 1159-1172). IREs bind iron-regulatory proteins (IRPs) and regulate iron homeostasis in eukaryotes. Two forms of the RNA secondary structure for IRE have been proposed in the literature (Gdaniec et al., Biochemistry, 1998, 37, 1505-1512). The stem-loop structure proposed differs in the structure of the internal loop disrupting the helix. The IRE secondary structure is most frequently shown with a C bulge on the 5′ side of the helix (FIG. 6A). An alternate structure has an asymmetrical internal loop at this same position with three unpaired bases on the 5′ side of the helix and a single C on the 3′ side (FIG. 2B). A single, highly specific RNAMotif descriptor can be written to capture both of these structural elements and identifies IREs in a number of iron-regulated transporters (Macke, Nucleic Acids Res., 2001, 29, 4724-4735). A less specific descriptor for this same structure element increases the number of false positives significantly but may also allow discovery of distantly related IREs over many species. A series of three descriptors of increasing generality over four experiments was used to test the ability of the EA to discover common IRE structures in ferritin mRNA sequences from a number of orthologous sequences.

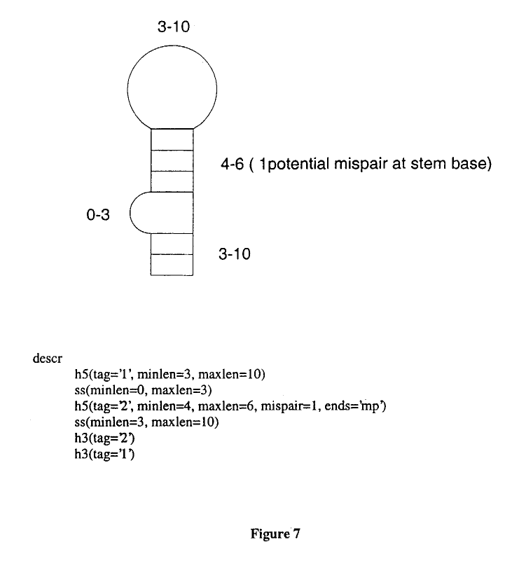

Seven full-length ferritin mRNA sequences (Homo sapiens, gi|507251; Sus scrofa, gi|286151; Cricetulus griseus, gi|191071; Gallus gallus, gi|2369860; Rana catesbeiana, gi|213691; Xenopus laevis, gi|214135; Drosophila melanogaster, gi|3559829) were obtained from GenBank. The descriptor shown in FIG. 7 was used to generate structure hits using RNAMotif. The number of hits for each experiment is given in Table 1.

TABLE 1

| Organism | Exp. 1 | Exp. 2 | Exp. 3 | Exp. 4 |
|---|---|---|---|---|
| Homo sapiens | 45 | 154 | 154 | 785 |
| Sus scrofa | 25 | 122 | 122 | 570 |
| Cricetulus griseus | 15 | 67 | 67 | 260 |
| Gallus gallus | 37 | 91 | 91 | 1228 |
| Rana catesbeiana | 9 | 100 | 100 | 142 |
| Xenopus laevis | 9 | 62 | 62 | 148 |
| Drosophila melanogaster | 15 | 137 | 137 | 554 |
| Cavia porcellus | | | 128 | |
| Oncorhynchus nerka | | | 57 | |
| Canis familiaris | | | 59 | |
| Danio rerio | | | 116 | |
| Mus musculus | | | 71 | |

The total number of hits for this experiment was 155. When each bin is allowed to contain one structure from each of the seven organisms and all possible combinations are allowed, there are 7.6×10⁸ possible bins in the search space.

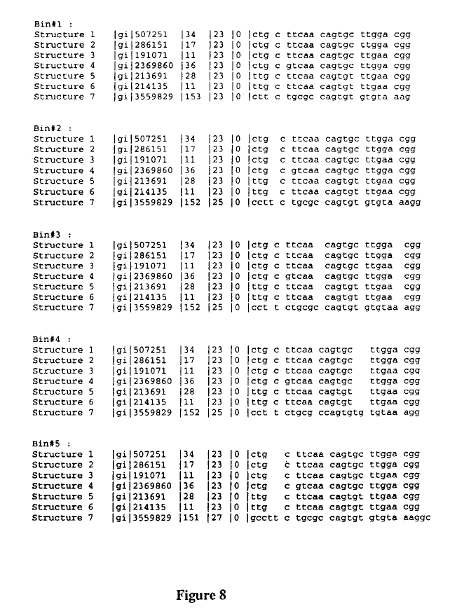

The evolutionary search examined only a fraction of the possible bins (1.4×10⁻⁵) before converging on a solution, which contained a set of structures that exactly matched the proposed IRE structure (FIG. 8). This was achieved by the 13th generation in <3 min. Exhaustive evaluation of all possible bin combinations for this experiment at the same rate of calculation would have required 125 days.

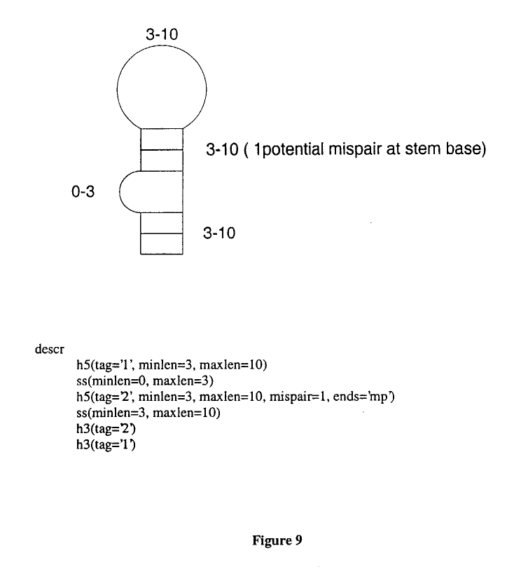

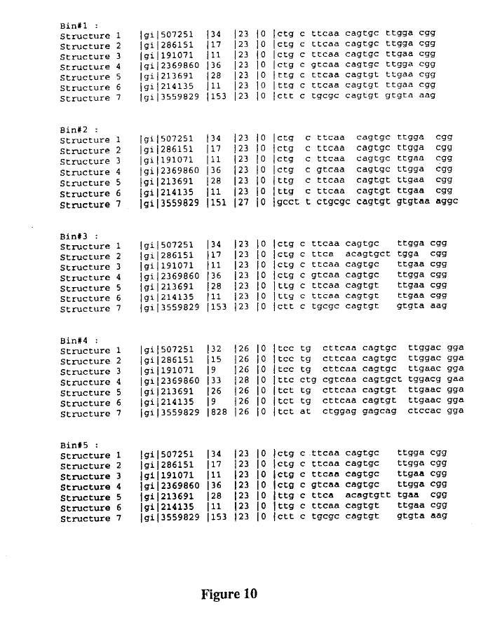

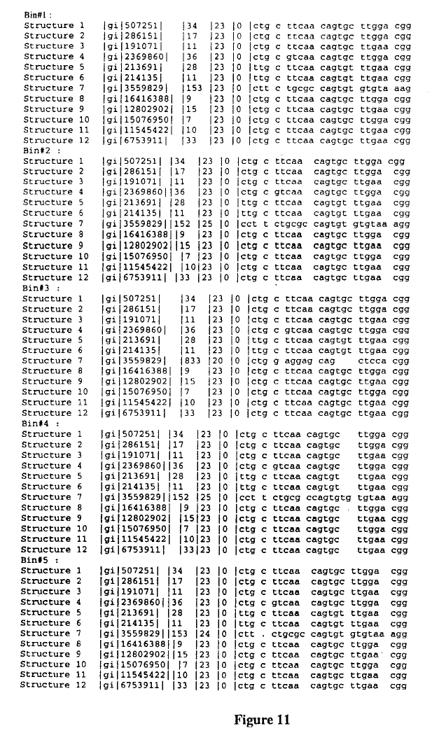

For the second experiment, the descriptor was altered to provide additional variation in the length of the upper tem (FIG. 9). The resulting number of hits for each organism in the sense strand for each mRNA is listed in Table 1. The total number of hits in the sense strand for this experiment was 733, representing 9.7×10¹³ possible bin combinations. A population of 40 parent bins and 20 offspring bins was used for 1000 generations of evolution. By generation 33, the best bin the population contained a set of structures identical to the IRE (FIG. 10). This calculation took 6 minutes on a set four Linux machines operating in parallel. This solution remained as the best solution in the population for the remaining 967 generations. To arrive a this best solution in generation 33, only 2.7×10⁻¹⁰ of the possible bins was evaluated. In a third experiment with this same descriptor, 5 additional ferritin mRNA sequences (Cavia porcellus, gi|16416388; Oncorhynchus nerka, gi|12802902; Canis familiaris, gi|15076950; Danio rerio, gi|11545422; Mus musculus, gi|6753911) were added to increase the size of the search space. The results of this experiment are shown in FIG. 11. The size of this space was 3.5×10²³, larger than that of experiment 1 by 15 orders of magnitude. With a population of 100 parents and 50 offspring operating in parallel on three Linux machines, 21 generations (1.1 hours) were required to converge on the correct solution. This represented a search of only 3.0×10⁻¹⁹ of the number of possible bins.

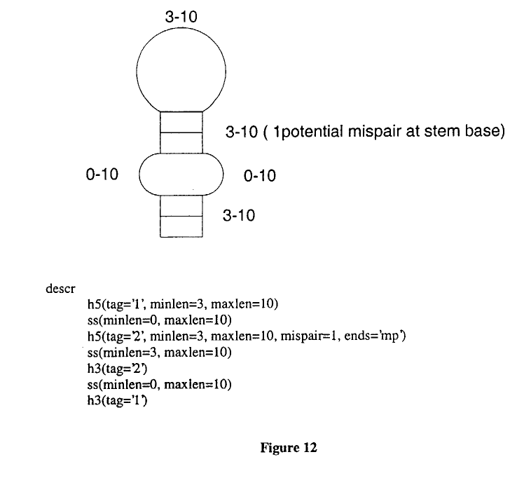

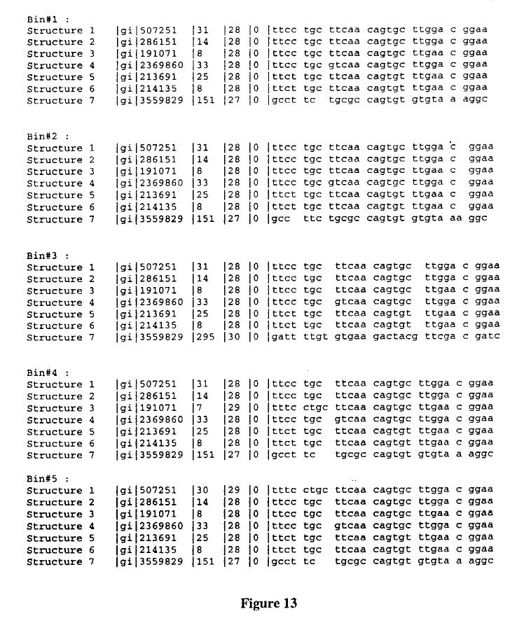

For the fourth IRE experiment, the descriptor was altered yet again to provide additional variation. The possibility of unpaired bases internal to the 3′-stem was incorporated into the descriptor with a minimum bulge length of zero and maximum of 10 unpaired bases. The number of unpaired bases on the 5′-stem was also increased to this same range (FIG. 12). This descriptor allowed for the possibility of both known forms of the ferritin IRE. The resulting number of hits in the sense strand for each mRNA is listing by organism in Table 1. The total number of hits in the sense strand for this experiment was 3867, representing 1.7×10¹⁸ possible bins when taking all potential combinations of structures. A population of 100 parent bins and 50 offspring bins was used for 1000 generations of evolution. By generation 115, the best bin in the population contained a set of structures identical to the alternative structure proposed for ferritin IRE (FIG. 13). This calculation tool took 3.0 hours on four Linux machines operating in parallel and 5.8×10⁵ total bins were evaluated. To arrive at this solution by generation 115, only 3.4×10⁻¹³ of the possible bins in the search space was evaluated.

### Example 2

SRP-RNA Domain IV Stem Loop Descriptor

The signal recognition particle (SRP) targets signal peptide-containing proteins to plasma membranes (prokaryotes) or the endoplasmic reticulum (eukaryotes) (Schmitz et al., RNA, 1999, 5, 1419-1429; Schmitz et al., Nature Struct. Biol., 1999, 6, 634-638). The SRP RNA (4.5S RNA in prokaryotes and 7S RNA in eukaryotes) is an essential component of the particle. A key portion of SRP is the domain IV stem-loop, which has been conserved from bacteria to mammals. Domain IV is the binding site for the protein component of the particle (Batey et al., Science, 2000, 287, 1232-1239). Key features of the domain IV stem-loop have been identified. These include two internal loops, a symmetrical loop near the top of the stem and a variable asymmetric loop closer to the base of the stem (Macke, Nucleic Acids Res., 2001, 29, 4724-4735). The helices are of varying length and the loop is typically one of two predominant types, either a tetraloop or hexaloop (FIG. 6C). Previous experimentation demonstrated that a single, highly specific RNAMotif descriptor is capable of finding SRP RNA domain IV structures in a wide range of bacterial genomes (Macke, Nucleic Acids Res., 2001, 29, 4724-4735). A less-specific descriptor would find all of these known SRPs but increase the number of false positives significantly. Evolutionary computation was used to search for common RNA structure with a series of less-specific SRP descriptors to test the resolution of the present invention.

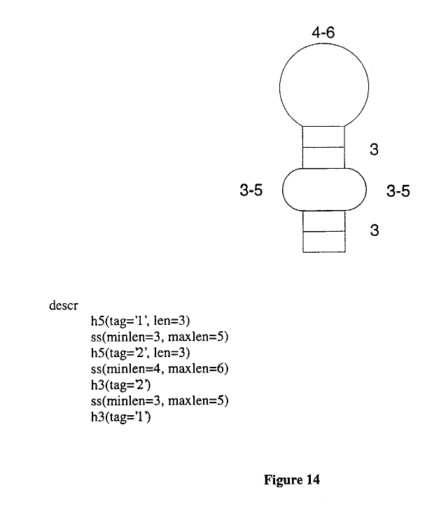

For the first SRP experiment, five full-length sequences for 4.5S/7S rRNA (Archaeoglobus fulgidus, gi|38795; Bacillus subtilis, gi|216348; E. coli, gi|42758; Homo sapiens, gi|177793; Methanococus voltae, gi|150042) were obtained from GenBank. The descriptor shown in FIG. 14 was used to screen these sequences for structures using RNAMotif. The resulting number of hits for each organism within the sense strand of these sequences is listed in Table 2.

TABLE 2

| Organism | Exp. 1 | Exp. 2 | Exp. 3 | Exp. 4 |
|---|---|---|---|---|
| Archeoglobus fulgidus | 15 | 69 | 200 | 724 |
| Bacillus subtilis | 12 | 62 | 374 | 520 |
| E. coli | 14 | 45 | 258 | 523 |
| Homo sapiens | 20 | 136 | 418 | 903 |
| Methanococcus voltae | 11 | 30 | 121 | 315 |

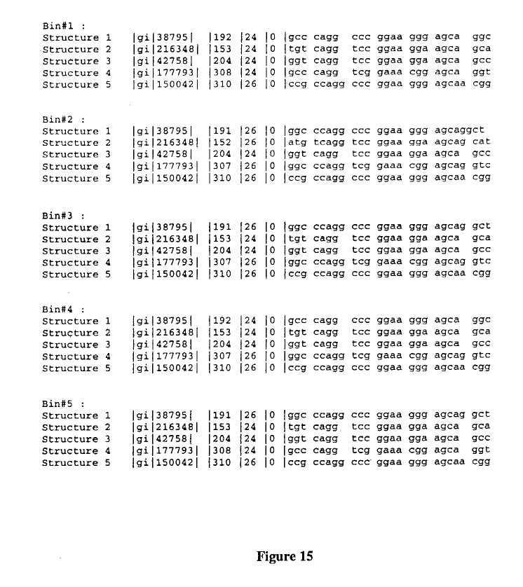

The total number of hits for this experiment was 72. When each bin contains one structure from each of the five organisms and all combinations are allowed, there are 5.5×10⁵ possible bins. A population of 80 parent bins and 40 offspring bins was used for 1000 generations of evolution. By generation 3, the best bin in the population contained a set of structures that matched the known SRP structure (FIG. 15). This calculation took 2.4 minutes on four Linux machines operating in parallel. This solution remained the best solution throughout the rest of evolution. In generation 3, only 1.7×10⁻² of the possible bins had been evaluated.

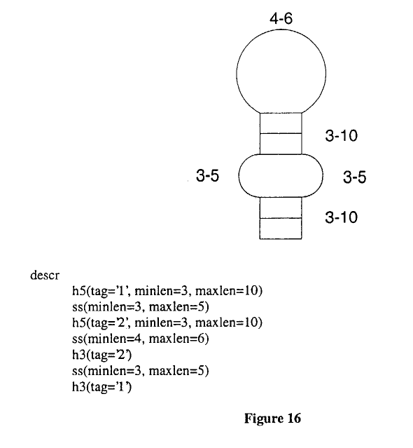

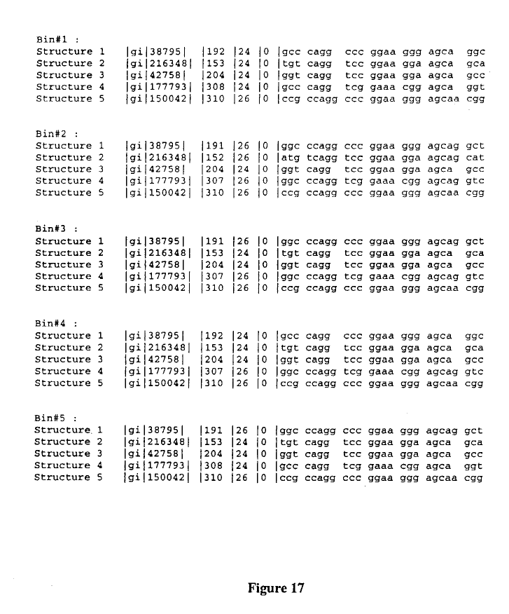

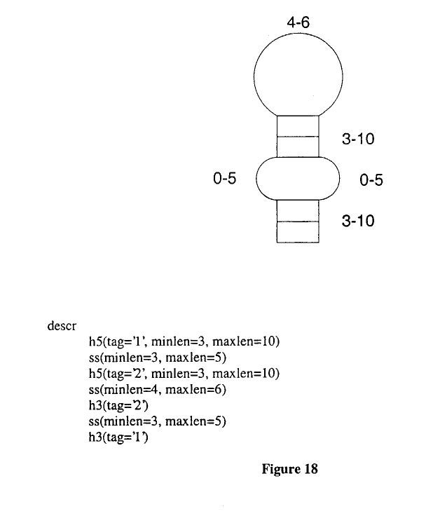

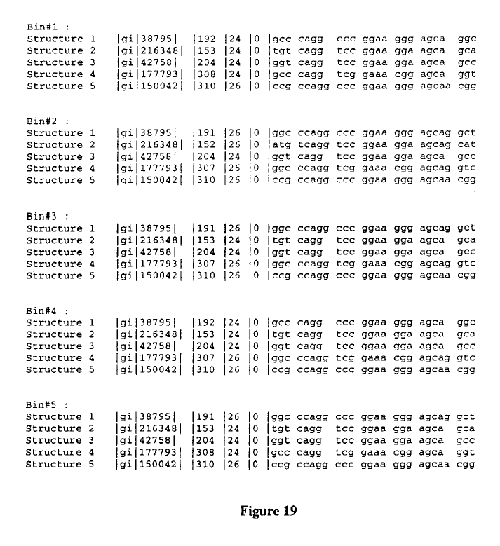

For the second SRP experiment, the descriptor was modified slightly to allow greater length variation in the stems (FIG. 16). This descriptor resulted in a space of 7.9×10⁸ possible bins. A population of 80 parents and 40 offspring operating on four Linux machines in parallel was able to generate a correct solution in 7 generations (4 minutes), representing a search of 2.8×10⁻⁵ of the possible bins (FIG. 17). In a third experiment, the internal loops of the descriptor were allowed to have additional length variation (FIG. 18). With this change, the number of possible bins increased significantly to a space of 9.8×10¹¹. Using a population of 80 parents and 40 offspring, the SRP structures was discovered in 27 generations (143 minutes) of evolution on four Linux machines (FIG. 19). This sampled only 8.8×10⁻⁸ of the possible bins.

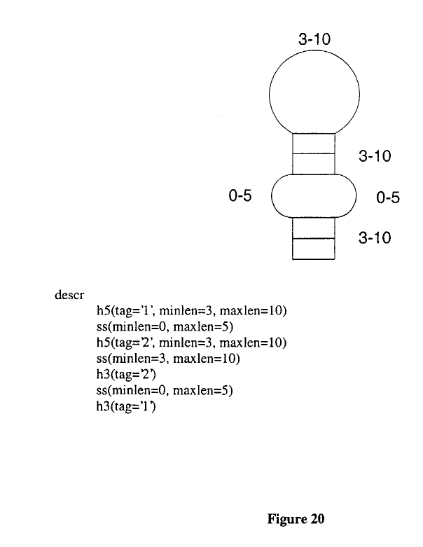

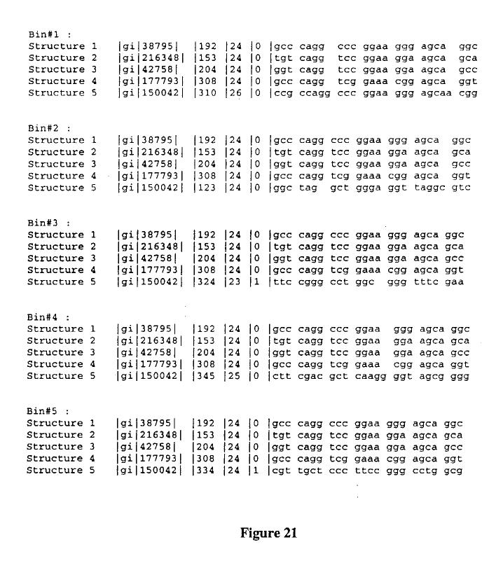

A fourth SRP experiment added variability to the stems, internal loop, and hairpin loop, further increasing the number of hits per organism (FIG. 20). A space of 5.6×10¹³ possible bins was generated with this descriptor. The RNAEvolve search of this space began with a population of 80 parent bins and 40 offspring bins was performed on four Linux machines in parallel. After 27 generations (12 minutes), the known SRP structure was discovered (FIG. 21). This represented a sampling of only 5.9×10⁻¹¹ of the possible bins.

Various modifications of the invention, in addition to those described herein, will be apparent to those skilled in the art from the foregoing description. Such modifications are also intended to fall within the scope of the appended claims. Each reference and GenBank reference cited in the present application is incorporated herein by reference in its entirety.

What is claimed is:

1. A method of detecting a conserved structure in an RNA sequence comprising the steps of:
a) placing at least two structures from a plurality of structures generated for at least two RNA sequences from at least two organisms into a parent group;
b) generating an offspring group from said parent group, wherein at least one structure is replaced in said parent group to generate said offspring group;
c) determining fitness of said parent and offspring groups;
d) comparing said fitness of said parent and offspring groups; and
e) selecting at least one group from said parent and offspring groups with the highest fitness, wherein said conserved structure in said RNA is present within said at least one group.

2. The method of claim 1 wherein steps b)-e) are repeated iteratively.

3. The method of claim 2 wherein iterations are stopped by a user-defined criteria.

4. The method of claim 3 wherein said user-defined criteria is number of generations, CPU time, clock time, or use of a statistical method to determine the appropriate number of generations.

5. The method of claim 4 wherein said statistical method determines when the expected change in fitness per generation is close to zero and past which further computation will not result in a large change in fitness but will take an unreasonable amount of time.

6. The method of claim 1 wherein said replaced structure is replaced with a structure from a different organism.

7. The method of claim 1 wherein said replaced structure is replaced with a structure from the same organism.

8. The method of claim 1 wherein said parent group comprises B structures, wherein said B is greater than one and less then or equal to the number of structures that were generated.

9. The method of claim 1 wherein said parent group comprises B structures, wherein said B is equal to the number of organisms.

10. The method of claim 1 wherein said parent group comprises at least one structure from each organism.

11. The method of claim 1 wherein said plurality of RNA structures are generated using RNAMotif.

12. The method of claim 1 wherein at least two structures are replaced in said parent group to generate said offspring group.

13. The method of claim 1 wherein said comparing the fitness of said groups is carried out by elitist selection.

14. The method of claim 1 wherein said comparing the fitness of said groups is carried out by tournament selection.

15. The method of claim 1 wherein said parent and offspring groups are treated as one evolving population.
